(19) United States
(12) Patent Application Publication
(10) Pub. No.: US 2012/0301559 A1
(43) Pub. Date: Nov. 29, 2012

(54) DIETARY REGIMENS USEFUL FOR MIMICKING CALORIC RESTRICTIONS

(76) Inventors: Sylvie Pridmore-Merten, St. Sulpice (CH); Ziad Ramadan, Manchester, MO (US)

(21) Appl. No.: 13/519,224

(22) PCT Filed: Jan. 5, 2011
(86) PCT No.: PCT/US11/00020

§ 371 Date: Aug. 15, 2012

Related U.S. Application Data
(60) Provisional application No. 61/335,448, filed on Jan. 6, 2010.

(51) Int. Cl. A61K 36/16; A61K 36/48; A61P 3/02; A23K 1/18

(52) U.S. Cl. 424/752; 426/2; 424/757; 424/766

(57) ABSTRACT

The invention provides dietary regimens useful mimicking caloric restriction in animals. The regimens use a first diet containing one or more dietary supplements suitable for mimicking caloric restriction when the animal is a young animal; a second diet containing one or more dietary supplements suitable for mimicking caloric restriction when the animal is an adult animal; and a third diet containing one or more dietary supplements suitable for mimicking caloric restriction when the animal is a senior animal; wherein the dietary supplements in the first diet, second diet, and third diet are not all be the same dietary supplements.

### CROSS REFERENCE TO RELATED APPLICATIONS

This application is a national stage application under 35 U.S.C. §371 of PCT/US2011/000020 filed Jan. 5, 2011, which claims priority to U.S. Provisional Application Ser. No. 61/335448 filed Jan. 6, 2010, the disclosures of which are incorporated herein by reference.

### BACKGROUND OF THE INVENTION

1. Field of the Invention

The invention relates generally to dietary regimens for animals and particularly to dietary regimens useful for mimicking caloric restriction in animals.

2. Description of Related Art

Caloric restriction and methods for mimicking caloric restriction are known to benefit animals. Caloric restriction or mimicking caloric restriction is known to delay the onset of age-related disease and increase the longevity of animals. US20080279786 discloses methods for extending lifespan and delaying the onset of age-related disease by administering oxaloacetate, oxaloacetic acid, oxaloacetate salt, alpha-ketoglutarate, and aspartate to an animal. US20080306157 discloses therapeutic interventions for mimicking the effect of caloric restriction by administering long chain free fatty acids or a composition resulting in endogenous release of long chain free fatty acids to an animal. US20060116330 discloses methods of mimicking the metabolic effects of caloric restriction by administration a glucose anti-metabolite such as 2-deoxy-D-glucose or mannoheptulose. US20040047896 discloses compositions for improving age-related physiological deficits and increasing longevity by mimicking the effects of caloric restriction on gene expression. Although these methods may be effective, there exists a need for new methods for mimicking caloric restriction and thereby delaying the onset of age-related disease in and increasing longevity of animals.

### SUMMARY OF THE INVENTION

It is, therefore, an object of the invention to provide dietary regimens useful for mimicking caloric restriction in animals.

It is another object of the invention to provide methods for mimicking caloric restriction in animals.

It is another object of the invention to provide methods for delaying the onset of age-related disease in animals.

It is another object of the invention to provide methods for increasing longevity of animals.

It is a further object of the invention to provide methods for promoting the health and wellness of animals.

It is another object of the invention to provide methods for improving quality of life for animals.

It is another object of the invention to provide methods for extending the prime for animals.

One or more of these other objects are achieved using dietary regimens comprising administering to the animal a first diet comprising a caloric restriction mimicking amount of one or more dietary supplements capable of mimicking caloric restriction when the animal is a young animal; a second diet comprising a caloric restriction mimicking amount of one or more dietary supplements capable of mimicking caloric restriction when the animal is an adult animal; and a third diet comprising a caloric restriction mimicking amount of one or more dietary supplements capable of mimicking caloric restriction when the animal is a senior animal; wherein the dietary supplements in the first diet, second diet, and third diet cannot all be the same dietary supplements.

Other and further objects, features, and advantages of the invention will be readily apparent to those skilled in the art.

### DETAILED DESCRIPTION OF THE INVENTION

### Definitions

The term “animal” means any animal that can benefit from dietary regimens for mimicking caloric restriction, e.g., a human, avian, bovine, canine, equine, feline, hicrine, lupine, murine, ovine, and porcine animals.

The term “companion animal” means any domesticated animal such as cats, dogs, rabbits, guinea pigs, ferrets, hamsters, mice, gerbils, horses, cows, goats, sheep, donkeys, pigs, and the like.

The term “young” means an animal of any age between infancy and adulthood. For example, “young” typically means an age of up to about 1 year for dogs; 1 year for cats; 3 years for horses; and 18 years for humans.

The term “adult” means an animal of an age after the completion of the juvenile growth and adolescent development stage until development of an increased risk of age-related disease. For example, “adult' typically means an age of from about 1 year to about 7 years for dogs; 1 to 8 years for cats; about 3 to 21 years for horses; and about 18 to 65 years for humans.

The term “senior” means an animal of an age having an increased risk for age-related disease but may or may not have obvious physical or behavioral characteristics of aging. For example, “senior” means an age of from about 7 years or older for dogs; 8 years or older for cats; 21 years or older for horses; and 65 years or older for humans.

The term “base diet” means a diet containing the nutrients necessary to support and maintain life for an animal.

The term “dietary supplement” means a compound or composition that is intended to be administered to an animal as an addition to a base diet.

The term “complete and nutritionally balanced” means a dietary composition or food that contains all known required nutrients for the intended recipient or consumer, in appropriate amounts and proportions, based for example on recommendations of recognized authorities in the field of animal nutrition. Such foods are therefore capable of serving as a sole source of dietary intake to maintain life or promote production, without the addition of supplemental nutritional sources. Complete and nutritionally balanced pet dietary compositions are widely known and widely used in the art.

The term “CR drugs” means any compound, composition, or drug useful for mimicking caloric restriction, excluding the dietary supplements of the invention.

The term “single package” means that the components of a kit are physically associated, in or with one or more containers, and considered a unit for manufacture, distribution, sale, or use. Containers include, but are not limited to, bags, boxes or cartons, bottles, packages of any type or design or material, over-wrap, shrink-wrap, affixed components (e.g., stapled, adhered, or the like), or combinations of any of the foregoing. For example, a single package kit may provide containers of individual compositions and/or food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain, for example, instructions on how to use the kit, or safety or technical information about one or more components of a kit. Examples of information that can be provided as part of a virtual kit include instructions for use; safety information such as material safety data sheets; poison control information; information on potential adverse reactions; clinical study results; dietary information such as food composition or caloric composition; diseases that effect and animal and their relationship to caloric restriction; and use, benefits, and potential side-effects or counter-indications for CR drugs.

The term “health and wellness of an animal” means the complete physical, mental, and social well being of the animal, not merely the absence of disease or infirmity.

The term “quality of life” means the ability to enjoy normal life activities.

The term “extending the prime” means extending the number of years an animal lives a healthy life and not just extending the number of years an animal lives, e.g., an animal would be healthy in the prime of its life for a relatively longer time.

All percentages expressed herein are by weight of the composition on a dry matter basis unless specifically stated otherwise. The skilled artisan will understand that the term “dry matter basis” means that an ingredient's concentration or percentage in a composition is measured or determined after any free moisture in the composition has been removed.

As used throughout, ranges are used herein in shorthand, so as to avoid having to set out at length and describe each and every value within the range. Any appropriate value within the range can be selected, where appropriate, as the upper value, lower value, or the terminus of the range.

As used herein and in the appended claims, the singular form of a word includes the plural, and vice versa, unless the context clearly dictates otherwise. Thus, the references “a”, “an”, and “the” are generally inclusive of the plurals of the respective terms. For example, reference to “an animal, “a method”, or “dietary supplement” includes a plurality of such “animals”, “methods”, or “dietary supplements”. Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Where used herein the term “examples,” particularly when followed by a listing of terms is merely exemplary and illustrative, and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved. Full citations for publications not cited fully within the specification are set forth at the end of the specification.

### The Invention

In one aspect, the invention provides dietary regimens useful for mimicking caloric restriction in animals. The regimens comprise administering to the animals a first diet comprising a caloric restriction mimicking amount of one or more dietary supplements capable of mimicking caloric restriction when the animal is a young animal; a second diet comprising a caloric restriction mimicking amount of one or more dietary supplements capable of mimicking caloric restriction when the animal is an adult animal; and a third diet comprising a caloric restriction mimicking amount of one or more dietary supplements capable of mimicking caloric restriction when the animal is a senior animal; wherein the dietary supplements in the first diet, second diet, and third diet cannot all be the same dietary supplements. The diets are administered to the animals during the corresponding stage in life, i.e., the first diet is administered to young animals, the second diet is administered to adult animals, and the third diet is administered to senior animals.

In various embodiments, the dietary supplements in each diet may be the same or may be different so long as the dietary supplements are not the same for all diets. In one embodiment, the dietary supplements in each diet are different dietary supplements. In another, the dietary supplements in two diets are different dietary supplements. In a further, the dietary supplements are the same in all diets except that (1) at least one diet is missing a dietary supplement that is in the other diets or (2) at least one diet has an additional dietary supplement that is not in the other diets.

The invention is based upon the discovery that no single dietary supplement or combination of dietary supplements is useful for mimicking caloric restriction throughout all stages of an animal's life (i.e., the young, adult, and senior stages of life) and that different dietary supplements or combinations of dietary supplements are needed to effectively mimic caloric restriction during different stages of an animal's life. Thus, an animal in the young, adult, and senior stages of life cannot be administered a particular supplement or combination of supplements that will mimic caloric restriction through all these stages of life. To effectively mimic caloric restriction, an animal must be administered particular dietary supplements or combinations of dietary supplements at each of these stages of life.

The dietary supplements used in the invention are any dietary supplements known to skilled artisans to mimic caloric restriction and that have been shown to be effective for a particular stage of life as described herein. The dietary supplements can be obtained or derived from any suitable source, e.g., natural or synthetic as appropriate for the particular dietary supplement. The supplements are administered to the animal in any amount (1) effective for mimicking caloric restriction and (2) not harmful to the animal, e.g., non-toxic. The selection of dietary supplements for and the amounts to be administered to a particular animal can be determined by skilled artisans. In preferred embodiments, the supplements are administered to the animal in amounts according to the recommended daily allowance (RDA) for the supplement and for the particular animal.

The dietary supplements may be in any form, e.g., solid, liquid, gel, tablets, capsules, powder, and the like. Preferably they are provided in convenient dosage forms. Most preferably, dietary supplements are incorporated into the animal's base diet. In some embodiments, the supplements are provided in bulk consumer packages such as bulk powders, liquids, gels, or oils, e.g., for administration in, on, or with one or more dietary compositions administered to the animal, e.g., the animal's food or in food items such as snacks, treats, supplement bars, beverages, and the like.

In certain embodiments, the dietary regimens comprise administering to the animal a first diet comprising a caloric restriction mimicking amount of at least one of Ginkgo biloba and L-carnitine when the animal is a young animal; a second diet comprising a caloric restriction mimicking amount of at least two of vitamin C, vitamin E, grape seed proanthocyanidin extract, and cysteine when the animal is an adult animal; and a third diet comprising L-carnitine when the animal is a senior animal. In one such embodiment, the dietary supplement in the first diet is Ginkgo biloba. In another, the dietary supplement in the first diet is L-carnitine. In a further, the dietary supplements in the first diet are a combination of Ginkgo biloba and L-carnitine. In various embodiments, the dietary supplements in the second diet are any two or three of various combinations of vitamin C, vitamin E, grape seed proanthocyanidin extract, and cysteine. In one such embodiment, the dietary supplements are grape seed proanthocyanidin extract and cysteine. In another, the dietary supplements are vitamin C, vitamin E, and grape seed proanthocyanidin extract. Preferably, the dietary supplements in such embodiments are vitamin C, vitamin E, grape seed proanthocyanidin extract, and cysteine.

The dietary supplements ginkgo biloba, L-carnitine, Vitamin C, Vitamin E, Seed proanthocyanidin extract, and cysteine used in the invention can be obtained or derived from any suitable source, e.g., natural or synthetic as appropriate for the particular dietary supplement. The supplements are administered to the animal in any amount (1) effective for mimicking caloric restriction and (2) not harmful to the animal, e.g., non-toxic. In preferred embodiments, the supplements are administered to the animal in amounts according to the recommended daily allowance (RDA) for the supplement and for the particular animal. Suitable amounts for particular supplements and particular animals can be determined by skilled artisans. In preferred embodiments, the ginkgo biloba is administered to the animal in amounts of from about 0.1 to about 10 mg/kg/day, preferably from about 0.5 to about 5 mg/kg/day, most preferably from about 1 to about 3 mg/kg/day. Alternatively, the ginkgo biloba is administered to the animal in amounts of from about 50 to about 500 mg/day, preferably from about 100 to about 300 mg/day, most preferably from about 120 to about 240 mg/day. The L-carnitine is administered to the animal in amounts of from about 0.05 to about 20 mg/kg/day, preferably from about 0.1 to about 10 mg/kg/day, most preferably from about 0.5 to about 5 mg/kg/day. Alternatively, from about 10 to about 1000 mg/day, preferably from about 20 to about 800 mg/day, most preferably from about 50 to about 500 mg/day. The vitamin C is administered to the animal in amounts of from about 0.5 to about 40 mg/kg/day, preferably from about 1 to about 30 mg/kg/day, most preferably from about 2 to about 20 mg/kg/day. Alternatively, from about 30 to about 3000 mg/day, preferably from about 50 to about 2000 mg/day, most preferably from about 100 to about 1500 mg/day. The vitamin E is administered to the animal in amounts of from about 0.1 to about 20 International Units per day (IU)/kg/day, preferably from about 0.5 to about 10 IU/kg/day, most preferably from about 1 to about 5 IU/kg/day. Alternatively, from about 10 to about 2000 IU/day, preferably from about 20 to about 1500 IU/day, most preferably from about 50 to about 800 IU/day. The seed proanthocyanidin extract is administered to the animal in amounts of from about 10 to about 1000 mg/kg/day, preferably from about 20 to about 600 mg/kg/day, most preferably from about 50 to about 300 mg/kg/day. The cysteine is administered to the animal in amounts of from about 6 to about 600 mg/kg/day, preferably from about 20 to about 500 mg/kg/day, most preferably from about 50 to about 400 mg/kg/day.

In certain embodiments, the dietary regimen comprises administering to an animal a complete and nutritionally balanced dietary composition comprising the dietary supplements needed for a particular stage of life, e.g., young, adult, or senior stages of life. In preferred embodiments, the animals are companion animals, preferably pets such as dogs or cats.

Preferably, the dietary regimen encompasses administering the dietary supplements to the animals throughout the animals life, i.e., while the animal is young, an adult, and a senior animal. However, the invention specifically includes regimens wherein the supplements are administered to the animals during part of one or more life stages, all of one or more life stages, or any combination thereof. For example, the dietary regimen can be started during the middle of adulthood and continued throughout the senior stage of life. Similarly the dietary regimen could be started at the beginning of the senior stage or at some time after the beginning of the senior stage. Further, the dietary regimen could be in place for only a fraction of a particular stage, although not preferred. In the preferred embodiment, the dietary regimen is in place during the young, adult, and senior stages of life. If circumstances dictate, the dietary regimen could be implemented at any time during life and continued throughout life.

The diets used in the dietary regimen are formulated as needed for a particular animal and desired administration. In various embodiments, the diets are formulated as human food diet, pet food diet, nutraceutical diet, or a pharmaceutical diet.

In various embodiments, the diets used in the regimen have at least one distinctive characteristic relative to at least one of the other diets. Such characteristics can be visual characteristics, olfactory characteristics, textural characteristics, size characteristics, shape characteristics, and the like. In one embodiment, the first, second, and third diets have different visual characteristics, e.g., color, shape, and the like. In another the first diet has a visual characteristic different from the second and third diet, the second diet has a shape characteristic different from the first and third diets, and the third diet has a different color characteristic from the first and second diets. In one embodiment, the three diets have a different color characteristic. In another, the three diets have a different shape characteristic. In a further, the three diets have a different size characteristic. Many such combinations can be created by a skilled artisan, e.g., various combinations of size, shape, and color. Such embodiments are useful for distinguishing the diets and their function in the regimen of the invention. Such embodiments are also useful for ensuring that the diets are compatible with the animals using the regimen, e.g., young animals may require smaller size dietary compositions because of their smaller size compared to when they are adults. Similarly, senior animals may require a softer textured diet compared to when they were adults.

In one embodiment, the dietary regimen further comprises administering one or more CR drugs to an animal in an amount effective for mimicking caloric restriction as part of the dietary regimen. CR drugs can be any CR drug known to skilled artisans. In various embodiments, the CR drugs are selected from the group consisting of resveratrol; metformin; endocannabinoid-1 receptor blockers; lipoic acids; 2-deoxy-D-glucose; mannoheptulose, leptin; peroxisome proliferator-activated receptor (PPAR) gamma modulators; and combinations thereof. CR Drugs also include compounds and compositions known to affect CR in animals such as those disclosed in US Patent Application Numbers US20080306157 and US20060116330.

The CR drugs are administered to the animal with the first diet alone, the second diet alone, the third diet alone, the first and second diet, the second and third diet, or any combination of the first, second, and third diets. In a preferred embodiment, the CR drugs are administered to the animal with all three diets.

A skilled artisan can determine the amount of CR drug to be administered to the animal based upon the recommended dosage for the drug given by its manufacturer and/or upon the animal's weight, species, age, health status, and the like. The CR drug can be administered by any suitable method, e.g., orally, and in any suitable form, e.g., solid, liquid, gel, tablets, capsules, powder, and the like. In preferred embodiments, the CR drug is administered as an ingredient in or on an animal's dietary composition, e.g., in the base diet along with the dietary supplements of the invention. The CR drug can be administered during the entire time a diet is administered to the animal or the CR drug can be administered for only part of the time a diet is administered to the animal.

In preferred embodiments, the diets of the invention comprise the dietary supplements according to the invention and a base diet comprising comestible ingredients and nutrients suitable for maintaining life for the animal. Preferably, the diet is a healthy diet that provides the energy and nutrients needed to maintain life and promote good health. In various embodiments, the diets contain protein, fat, carbohydrate, fiber, minerals, vitamins, and other ingredients and nutrients suitable for consumption by the animal. Such diets are known to skilled artisans. Preferably, the diet is a complete and balanced diet, e.g., a diet suitable for pets such as dogs and cats. Such diets can contain CR drugs as described herein. The diets may be specially formulated for the intended recipients or consumers, such as for adult animals or for senior or young animals. For example, a food composition adapted for puppies or kittens or adapted for active, pregnant, lactating, or aging animals can be prepared. In general, specialized compositions will comprise energy and nutritional requirements appropriate for animals at different stages of development or age.

A dietary regimen useful for mimicking caloric restriction has many beneficial effects for an animal. For example, mimicking caloric restriction affects longevity, the onset of age-related disease, health and wellness, quality of life, and the prime for an animal. Therefore, in one aspect, the invention provides methods for one or more of (1) mimicking caloric restriction in an animal, (2) delaying the onset of age-related disease in an animal, (3) increasing longevity of an animal, (4) promoting the health and wellness of an animal, (5) improving quality of life for an animal, and (6) extending the prime for an animal. The methods comprise mimicking caloric restriction in the animal by administering to the animal a first diet comprising a caloric restriction mimicking amount of one or more dietary supplements capable of mimicking caloric restriction when the animal is a young animal; a second diet comprising a caloric restriction mimicking amount of one or more dietary supplements capable of mimicking caloric restriction when the animal is an adult animal; and a third diet comprising a caloric restriction mimicking amount of one or more dietary supplements capable of mimicking caloric restriction when the animal is a senior animal; wherein the dietary supplements in the first diet, second diet, and third diet cannot all be the same dietary supplements. In various embodiments, the first, second, and third diets comprise the dietary supplements Ginkgo biloba, L-carnitine, vitamin C, vitamin E, grape seed proanthocyanidin extract, and cysteine in combinations and amounts as described herein.

In a further aspect, the invention provides kits suitable for implementing and maintaining a dietary regimen that mimics caloric restriction to an animal. The kits comprise in separate containers in a single package or in separate containers in a virtual package, as appropriate for the kit component, at least one of (A) a first diet comprising a caloric restriction mimicking amount of one or more dietary supplements capable of mimicking caloric restriction when the animal is a young animal; (B) a second diet comprising a caloric restriction mimicking amount of one or more dietary supplements capable of mimicking caloric restriction when the animal is an adult animal; and (C) a third diet comprising a caloric restriction mimicking amount of one or more dietary supplements capable of mimicking caloric restriction when the animal is a senior animal; and at least one of (a) instructions for how to administer the diets to an animal, particularly to comply with the dietary regimen; (b) one or more CR drugs; (c) instructions for how to administer CR drugs to an animal, particularly to augment the dietary regimen; and (d) one or more devices useful for administering the diets to an animal, e.g., a bowl, spoon, spatula, and the like.

In a further aspect, the invention provides kits suitable for making one or more of the diets of the invention. The kits comprise one or more dietary supplements known to mimic caloric restriction during at least one stage of an animal's life and at least one of (a) one or more comestible ingredients; (b) instructions for how to combine the dietary supplements and comestible ingredients to prepare one or more of diets of the invention; (c) one or more CR drugs; (d) instructions for how to administer CR drugs to an animal, particularly to as a dietary component; and (d) one or more devices useful for administering the diets to an animal, e.g., a bowl, spoon, spatula, and the like.

When a kit comprises a virtual package, the kit is limited to instructions in a virtual environment in combination with one or more physical kit components.

The kit components may each be provided in separate containers in a single package or in mixtures of various components in different packages. In preferred embodiments, the kits comprise the diets, dietary supplements, and other components in various combinations. For example, a kit could comprise a diet (e.g., the diet in A above) in one container and one or more CR drugs in another container. Or, a kit could comprise a diet (e.g., the diet in B above), instructions for administering the diet to an animal on a website, and a food bowl accompanying the diet. Similarly, a kit could comprise a mixture of one or more dietary supplements in one container and one or more dietary supplements or other components in a separate container, e.g., Ginkgo biloba in one container, L-carnitine in another container, and a complete and balanced food in another container. Similarly, the kit could comprise a mixture of vitamin C and grape seed proanthocyanidin extract in one container and vitamin E in another container, both attached to a bag containing a complete and balanced pet food. Other such combinations can be produced by the skilled artisan based upon the characteristics of the ingredients; their physical and chemical properties and compatibilities; and the life stage and type of the animal. The kits contain the dietary supplements and other components in amounts sufficient for mimicking caloric restrictions as described herein. Typically, dietary supplements and the other suitable kit components are admixed just prior to consumption by an animal.

In another aspect, the invention provides a means for communicating information about or instructions for one or more of (1) mimicking caloric restriction in an animal, (2) delaying the onset of age-related disease in an animal, (3) increasing longevity of an animal, (4) promoting the health and wellness of an animal, (5) improving quality of life for an animal, and (6) extending the prime for an animal; and (7) using the kits of the invention for the benefit of the animals. The means comprises one or more of a physical or electronic document, digital storage media, optical storage media, audio presentation, audiovisual display, or visual display containing the information or instructions. Preferably, the means is selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, the invention provides a package comprising a diet of the invention and a label affixed to the package containing a word or words, picture, design, acronym, slogan, phrase, or other device, or combination thereof, that indicates that the contents of the package contains a diet suitable for one or more of (1) mimicking caloric restriction in an animal, (2) delaying the onset of age-related disease in an animal, (3) increasing longevity of an animal, (4) promoting the health and wellness of an animal, (5) improving quality of life for an animal, and (6) extending the prime for an animal. Typically, such device comprises the words “mimics caloric restriction”, “increases longevity, “promotes health and wellness”, “improves quality of life” or an equivalent expression printed on the package. Any package or packaging material suitable for containing the diets is useful in the invention, e.g., a bag, box, bottle, can, pouch, and the like manufactured from paper, plastic, foil, metal, and the like. In a preferred embodiment, the package contains diet adapted for a particular animal such as a human, canine or feline, as appropriate for the label, preferably a companion animal diet.

In another aspect, the invention provides dietary compositions useful for useful for mimicking caloric restriction in animals. For young animals, the dietary compositions comprise a caloric restriction mimicking amount of one or more dietary supplements selected from the group consisting of Ginkgo biloba, L-carnitine, or combinations thereof. For adult animals, the dietary compositions comprise a caloric restriction mimicking amount of at least two or more dietary supplements selected from the group consisting of vitamin C, vitamin E, grape seed proanthocyanidin extract, and cysteine. The dietary supplements can be combined in any and all combinations of two or three dietary supplements. In a preferred embodiment, the diets contain all four dietary supplements, i.e., vitamin C, vitamin E, grape seed proanthocyanidin extract, and cysteine. For senior animals, the dietary compositions comprise a caloric restriction mimicking amount of L-carnitine.

In another aspect, the invention provides multi-pack packages. The multi-pack packages comprise a plurality of containers containing one or more dietary supplements useful in the invention arranged in an array and one or more devices for retaining the containers in the array. In some embodiments, the multi-pack packages have one or more handles affixed to the packages to facilitate handling and transporting the packages. In various embodiments, the devices are boxes made from paper, plastic, polymers, and combinations thereof. In others, the devices are systems of connected plastic rings affixed to each of the containers. In still others, the devices are wrappings of plastic of similar materials, e.g., twelve cans stacked in an array and wrapped in plastic. In preferred embodiments, the multi-pack packages further comprise one or more indicia describing the contents of the containers in the packages, e.g., labels, printing on the packages, stickers, and the like. In other embodiments, the devices further comprise one or more windows that permit the package contents to be viewed without opening the multi-pack package. In some embodiments, the windows are a transparent portion of the devices. In others, the windows are missing portions of the devices that permit the containers to be viewed without opening the multi-pack package. In a preferred embodiment, the muti-pack package has a label affixed to the package containing a word or words, picture, design, acronym, slogan, phrase, or other device, or combination thereof, that indicates that the contents of the package contains dietary supplements useful in a diet suitable for one or more of (1) mimicking caloric restriction in an animal, (2) delaying the onset of age-related disease in an animal, (3) increasing longevity of an animal, (4) promoting the health and wellness of an animal, (5) improving quality of life for an animal, and (6) extending the prime for an animal.

### EXAMPLES

The invention can be further illustrated by the following examples, although it will be understood that the examples are included merely for purposes of illustration and are not intended to limit the scope of the invention unless otherwise specifically indicated.

### Example 1

Experimental Design: C57BL/6J male mice were obtained at 9 weeks of age to constitute a primary colony. Mice belonging to a reserve colony were fed a control diet (A) until they entered the study. At 3 months (young), 12 months (adult), and 21 months (senior) of age respectively, a small number of mice were switched to one of the experimental diets for short-term interventions lasting 3 months. The long-term interventions were initiated at 3 months of age and lasted 21 months. Mice were housed individually, submitted to 12 hours inverted light and dark cycles, and had free access to water. With the exception of caloric restricted mice, all dietary groups were fed ad libitum. Caloric restricted mice were fed once a day concomitantly with the beginning of the dark cycle. Health status was monitored twice daily on weekdays and once during the weekend. Weight and food consumption were recorded weekly for each animal. Mice were kept in a certified specific pathogen free (SPF) conditions. At the end of each intervention mice were sacrificed. Sacrifice took place in the first half of the dark phase; plasma was collected in the post-prandial (fed) state.

Diets: A control diet (A) contained soy and whey proteins, carbohydrates, fat, and other ingredients as shown in Table 1 and Table 2. Diet A served as a base diet to which different various different dietary supplement were added to produce the other diets used in the experiments. Diet C included a cocktail of antioxidants comprising vitamin C, vitamin E, grape seed proanthocyanidin extract, and cysteine (GSPE). Diet D included L-carnitine and the cocktail of antioxidants. Diet F was supplemented with L-carnitine and diet E with Ginkgo biloba extract. The compositions are given in Table 3. For caloric restriction (diet B) all energy sources, i.e., fat, starch, and sucrose, were reduced to provide 67% of the daily calorie consumption of the control group while providing 100% of necessary proteins, minerals and vitamins. Mice groups were named after the diet they consumed (A, B, C, D, E and F). Groups enrolled in long-term interventions were labeled with L (long), as shown in Table 4.

Sample Collection and Preparation: Blood was collected with ethylendiamintetraacetate (EDTA) as anticoagulant after mice decapitation. Plasma was immediately separated by centrifugation and stored at −80° C. prior to analysis by proton nuclear magnetic resonance (1H NMR) spectroscopy. For NMR analysis, 20 μL of plasma were added to 20 μL of deuterated phosphate buffer (0.2 M Na2HPO4/0.2M NaH2PO4; pH 7.4; 4.5 mM sodium azide). Deuterium oxide (10%) was used as locking substance. 10 μl of the mixture were transferred into 1 mm NMR tubes using a Gilson robot. The listing of the samples analyzed for each group is shown in Table 4.

NMR Data Analysis: Plasma samples were measured on a Bruker DRX-600 NMR spectrometer equipped with a 1 mm TXI Probe at a temperature of 300K and an automatic sample changer (Brucker Biospin, Germany). 1H NMR plasma spectra were acquired using the Carr-Purcell-Meiboom-Gill (CPMG) sequence (D-90°-(τ-180°-τ)n-acquisition) at a temperature of 300K. The spin echo loop time (2-τn) was adjusted to 142 ms and a total of 512 scans were acquired. Typical acquisition parameters included 32 K data points, a spectral width of 9615 Hz and a relaxation delay (D) of 2 s. All spectra were acquired with the same receiver gain. Before Fourier transformation, free induction decays were multiplied by an exponential weighting function corresponding to a line broadening of 1 Hz. The spectra were manually corrected for phase and baseline distortions using XWinNMR 3.5 software (Bruker Biospin, Rheinstetten, Germany). The spectra were referenced to the methyl resonance of lactate at δ 1.33 ppm. The 1H NMR spectra were imported over the range δ 0.2-10 ppm using Matlab (version 6.5.1 Release 13, The Mathworks, Mass., USA) in-house developed routines. The regions containing the water resonance (δ 4.74-4.88 ppm) and EDTA (δ 3.36 and 3.69 ppm) were removed.

Statistical Analysis: The spectra were normalized to a constant sum of all intensities within the specified range and auto-scaled prior to chemometric analyses. The multivariate pattern recognition techniques used in this study were based on the principal component analysis (PCA) and orthogonal-projection to latent structure discriminant analysis (O-PLS-DA) using the software package SIMCA-P+ (version 11.0, Umetrics AB, Umea, Sweden) and an in-house developed MATLAB routines. PCA was first applied to NMR variables for global analysis of metabolites variance in the plasma profiles. Additional detailed classification studies were performed using O-PLS-DA to exclusively focus on the effects of caloric restrictin (CR), diets and aging. O-PLS-DA provides a way to filter out metabolic information (NMR spectral data, coded as X matrix) that is not correlated to the pre-defined classes (age, treatments, coded as dummy Y matrix). Influential variables that are therefore correlated to the group separation are identified using the variable coefficients according to a previously published method by Cloarec and coworkers. The weight of a variable in the discrimination is given its correlation coefficient (r). The standard 7-fold cross validation method was used to test the validity of the model. The classification accuracy of the O-PLS-DA model was established from the prediction-set samples in the 7-fold cross-validation, using a decision-rule based on the largest predicted Y value. To test the validity of the model against over-fitting, the goodness of fit (R2X and R2Y) and predictability (Q2Y) values of O-PLS-DA models were computed and reported in Table 5 and Table 6.

1H NMR metabolic profiling of plasma was used to simultaneously measure the relative concentrations of metabolites involved in different pathways to assess the overall effect of age, CR, and other dietary interventions on the plasma metabolome of mice. The results show that the metabolites significantly modulated by age or diets in postprandial phase together with the chemical shift of representative signals are: glucose (δ 3.84, 4.64, 5.24), lactate (δ 1.33, 4.13), citrate (δ 2.52, 2.64), pyruvate (δ 2.38), 3-D-hydroxybutyrate (δ 1.20), N-acetyl glycoproteins (δ□ 2.08), several amino acids (alanine (δ 1.49), valine (δ 1.05), isoleucine (δ 1.02), lysine (δ 1.73), taurine (δ 3.27, 2.41), methionine (δ 2.14), threonine (δ 4.25), tyrosine (δ 6.91), phenylalanine (δ 7.43), lysine (δ 3.03), histidine (δ 7.80), glutamine (δ 2.45), and blood plasma lipids (δ 0.853, 0.870, 0.882. 1.3, 5.35).

PCA was performed on the whole set of plasma 1H NMR spectra generated from mice of all ages submitted to the different diets. The first two principal components explained 26% of the total metabolic variance. The PCA scores showed that the plasma metabolic profiles from young and senior mice clustered together irrespective of the dietary interventions. The mature mice diverge from the main trend by spanning along the first principal component. The plasma of mature mice was characterized by lower levels of plasma glucose, lactate, pyruvate, 3-D-hydroxybutyrate and lipids and higher levels of amino acids when compared to young and senior animals.

Comparisons of the metabolic profiles using cross validated O-PLS-DA were applied to maximize the discrimination between sample groups focusing on differences according to age-dependant metabolic variations, CR, and dietary interventions. A first O-PLS-DA approach was performed to model the aging trajectory in the different dietary intervention groups. In all cases, including the control group, the plasma profiles were significantly separated according to age, as shown by the positive value of Q2Y in Table 5, and the samples were distributed from young to senior mice along the first predictive component. CR nice and mice supplemented with antioxidant cocktail (diet C) showed a different trajectory marked by higher metabolic variations in blood plasma of mature mice that deviated from both young and senior mice. These samples were characterized by lower glucose and elevated amino acid levels. The impact of the age of initiation on the efficacy of the dietary interventions and the metabolic changes induced by each diet was further assessed for young, adult, and senior animals.

Age-related plasma metabolic changes were assessed in the control groups at three different stages of life, i.e., 6, 15 and 24 months of age. The data is shown in Table 7. Between the ages of 6 to 15 months, metabolic profiling of blood plasma showed age-dependent increase of the plasma levels for several amino acids (isoleucine, serine, valine, alanine, methionine, lysine, threonine, tyrosine, phenylalanine and histidine. The metabolism of lipids was also modified as observed with higher levels of triglycerides rich (TG-rich) lipids and unsaturated fatty acids and decreased levels of 3-D-hydroxybutyrate and phospholipids. Metabolites of the glycolysis/gluconeogenesis pathway, i.e., pyruvate, lactate, and glucose were also decreased with age.

In the next life stage, comparing control mice aged 24 months to their younger counterpart of 15 months, the panel of metabolites exhibited a close mirror image of the previous phase with an increase in metabolites of the glycolysis/gluconeogenesis pathway (pyruvate, citrate and lactate). Amino acids levels were, for most, decreased with the exception of histidine and methionine exhibiting no changes and for glutamine, which plasma level increased. Unsaturated fatty acids and TG-rich lipids were significantly decreased and phospholipids increased (Table 7).

For all short dietary supplementations, plasma metabolites were measured after a 3 month treatment starting at 3, 12 and 21 months of age, thus assessing the effect of the treatment when given in young, mature and senior animals. To assess the impact of each treatment at each age, the plasma metabolite profiles obtained after treatment were compared to those of the control mice at the same age. The data is shown in Table 8.

When compared to their respective age-matched control, all CR mice exhibited a consistent and significant increase in circulating amino acids isoleucine, valine and tyrosine, as well as lactate. Glucose levels were consistently reduced in all CR mice. In addition 3-D-hydroxybutyrate, alanine, and threonine were only consistently modulated when CR was initiated before mid-life (young and mature). Metabolites of the lipid metabolism were not regulated in a similar fashion in the different CR interventions. In plasma of young CR mice, levels of phospholipids and unsaturated fatty acids were increased whilst TG-rich lipids and 3-D-hydroxybutyrate levels were reduced when compared to the control mice. In mature CR mice, blood plasma lipids and 3-D-hydroxybutyrate were lower when compared to the control mice. When CR was initiated in senior mice, only plasma unsaturated lipids were increased. In long-term CR, blood plasma levels of lipids were decreased and the level of 3-D-hydroxybutyrate increased (Table 8).

### Listing of Tables

TABLE 1
Composition of Control and Caloric Restricted Diet

| Ingredients | | Diet A (Control) | Diet B (CR) |
|---|---|---|---|
| Soybean oil | | 11.0 | 11.0 |
| Protein Mix | Soya | 15.0 | 21.5 |
| | Whey | 5.0 | 7.2 |
| Cornstarch | | 49.2 | 38.7 |
| Sucrose | | 10.0 | 7.8 |
| Cellulose | | 5.0 | 7.2 |
| Mineral Mix 93M | | 3.5 | 5.0 |
| Vitamin Mix 93vx | | 1.0 | 1.4 |

TABLE 2
Amino Acid Composition of the Control Diet

| Amino Acids | Soya | WP | Soya:WP (3:1) |
|---|---|---|---|
| Arginine | 1.48 | 0.34 | 1.19 |
| Histidine | 0.49 | 0.34 | 0.45 |
| Isoleucine | 0.79 | 1.12 | 0.87 |
| Leucine | 1.49 | 1.84 | 1.58 |
| Valine | 0.83 | 1.12 | 0.90 |
| Threonine | 0.70 | 1.31 | 0.86 |
| Lysine | 1.15 | 1.66 | 1.28 |
| Methionine | 0.22 | 0.32 | 0.24 |
| Cysteine | 0.25 | 0.43 | 0.30 |
| Phenylalanine | 0.97 | 0.54 | 0.86 |
| Tyrosine | 0.76 | 0.67 | 0.73 |
| Tryptophan | 0.20 | 0.31 | 0.23 |
| Glutamic acid | 3.58 | 3.01 | 3.44 |
| Alanine | 0.77 | 0.85 | 0.79 |
| Serine | 0.99 | 0.90 | 0.97 |
| Proline | 0.99 | 1.04 | 1.00 |
| Aspartic acid | 2.16 | 1.98 | 2.12 |
| Glycine | 0.77 | 0.31 | 0.66 |
| Valine | 0.85 | 1.06 | 0.90 |

TABLE 3
Dosing and Composition of Experimental Diets

| Supplement | Diet C | Diet D | Diet E | Diet F |
|---|---|---|---|---|
| L-carnitine | — | 0.30 | — | 0.30 |
| Vitamin C | 0.190 | 0.190 | — | — |
| Vitamin E | 0.045 | 0.045 | — | — |
| Grape Seed | 0.075 | 0.075 | — | — |
| Extract | | | | |
| Cysteine | 0.400 | 0.400 | — | — |
| Ginkgo | — | — | 0.0375 | — |

TABLE 4
Metabolomic Analyses

| Diet (group) | Young | Adult | Senior |
|---|---|---|---|
| Control (A) | n = 9 | n = 9 | n = 9 |
| CR (B) | n = 3 | n = 8 | n = 7 |
| Antioxidants (C) | n = 7 | n = 6 | n = 7 |
| Antioxidants + L-carnitine (D) | n = 7 | n = 5 | n = 6 |
| Ginkgo (E) | n = 4 | n = 7 | n = 8 |
| L-carnitine (F) | n = 6 | n = 7 | n = 9 |
| CR long (BL) | | | n = 7 |
| Antioxidants + L-carnitine long (DL) | | | n = 7 |

TABLE 5
Summary of the Generated O-PLS-DA Models for Discrimination
of Aging-Dependent Metabolic Changes for each Treatment

| Diet | R²X | R²Y | Q² |
|---|---|---|---|
| A | 0.43 | 0.96 | 0.39 |
| B | 0.37 | 0.99 | 0.36 |
| C | 0.49 | 0.99 | 0.29 |
| D | 0.31 | 0.97 | 0.25 |
| E | 0.42 | 0.98 | 0.33 |
| F | 0.40 | 0.97 | 0.46 |

TABLE 6
Summary of the Generated O-PLS-DA Models for Pair Wise
Class Discrimination between Diets at Different Ages

| Group | R²X | R²Y | Q² |
|---|---|---|---|
| Age | | | |
| A-Y-A | 0.46 | 0.99 | 0.34 |
| A-O-A | 0.45 | 0.99 | 0.57 |
| Young | | | |
| B/A | 0.43 | 0.99 | 0.68 |
| C/A | 0.47 | 0.99 | 0.64 |
| D/A | 0.43 | 0.99 | 0.48 |
| E/A | 0.55 | 0.99 | 0.23 |
| F/A | 0.53 | 0.99 | 0.60 |
| Adult | | | |
| B/A | 0.51 | 0.97 | 0.64 |
| C/A | 0.53 | 0.97 | 0.23 |
| D/A | 0.50 | 0.96 | 0.05 |
| E/A | 0.46 | 0.97 | 0.02 |
| F/A | 0.51 | 0.95 | 0.23 |
| Senior | | | |
| B/A | 0.32 | 0.99 | 0.44 |
| BL/A | 0.47 | 0.99 | 0.68 |
| C/A | 0.26 | 1.00 | 0.21 |
| D/A | 0.27 | 1.00 | 0.23 |
| ADL | 0.26 | 1.00 | −0.29 |
| AE | 0.29 | 1.00 | 0.05 |
| AF | 0.41 | 0.99 | 0.27 |

TABLE 7

| | A—Aging Related | | B—Age Specific | | | |
|---|---|---|---|---|---|---|
| | Metabolic Changes | | Metabolic Effect of CR | | | |
| | M/Y | M/O | Y | A | O | L |
| Lipid | −0.4407 | 0.488 | 0.2798 | −0.3481 | −0.0634 | −0.3058 |
| (0.85 L) | | | | | | |
| Lipid (0.870) | 0.1426 | −0.1456 | −0.2646 | −0.589 | −0.1164 | 0.2065 |
| Lipid (0.882) | 0.5305 | −0.6665 | −0.4064 | −0.2826 | −0.0654 | 0.6625 |
| Lipid (1.3) | −0.0592 | 0.0652 | 0.5563 | 0.0793 | 0.2691 | 0.4827 |
| Unsaturated | 0.2381 | −0.2683 | 0.4809 | −0.1346 | 0.3903 | 0.5094 |
| Lipids (5.35) | | | | | | |
| 3-D- | −0.4382 | −0.2062 | −0.4614 | −0.4715 | −0.0342 | −0.3542 |
| Hydroxy- | | | | | | |
| butyrate | | | | | | |
| Citrate | 0.17 | 0.2032 | 0.0294 | 0.1902 | −0.1199 | −0.6456 |
| Lactate | −0.5206 | 0.2976 | 0.5573 | 0.3873 | 0.325 | 0.304 |
| Pyruvate | −0.5419 | 0.7429 | 0.0595 | 0.1781 | −0.1649 | −0.1064 |
| Alanine | 0.3577 | −0.2334 | 0.459 | 0.7018 | 0.1498 | 0.512 |
| Glutamine | −0.2705 | 0.6229 | 0.8346 | −0.206 | −0.0389 | −0.2677 |
| N-acetyled- | 0.6531 | −0.3662 | −0.2991 | 0.5279 | −0.4877 | −0.154 |
| Glycoprotein | | | | | | |
| Histidine | 0.2227 | 0.0806 | −0.0747 | 0.2439 | −0.423 | −0.1372 |
| Isoleucine | 0.3507 | −0.3781 | 0.7323 | 0.6442 | 0.5641 | 0.7706 |
| Lysine | 0.3224 | −0.4341 | 0.1145 | 0.4945 | 0.3771 | 0.696 |
| Methionine | 0.2906 | −0.1118 | 0.2273 | 0.2657 | −0.2969 | 0.0724 |
| Phenyl- | 0.2567 | −0.3543 | 0.0186 | 0.3032 | 0.1526 | 0.5845 |
| alanine | | | | | | |
| Taurine | 0.1483 | −0.1006 | −0.3313 | 0.0929 | −0.2383 | 0.2083 |
| Threonine | 0.4208 | −0.2482 | 0.4466 | 0.5097 | −0.2272 | 0.2874 |
| Tyrosine | 0.4242 | −0.4155 | 0.2934 | 0.4757 | 0.3347 | 0.5738 |
| Valine | 0.3304 | −0.3137 | 0.9 | 0.7675 | 0.6138 | 0.9021 |
| Glucose | −0.3086 | 0.1312 | −0.5492 | −0.6299 | −0.3663 | −0.7267 |

TABLE 8

| | CR | Diet C | Diet D | Diet E | Diet F |
|---|---|---|---|---|---|
| | A—In Young Mice vs. Normal Aging Mice | | | | |
| Lipid (0.85 L) | 0.2798 | 0.0342 | 0.05 | −0.3743 | −0.6058 |
| Lipid (0.870) | −0.2646 | −0.0943 | −0.2257 | −0.1546 | −0.5362 |
| Lipid (0.882) | −0.4064 | 0.1944 | −0.0202 | 0.3595 | 0.2441 |
| Lipid (1.3) | 0.5563 | 0.5977 | 0.3923 | 0.4605 | 0.211 |
| Unsaturated Lipids (5.35) | 0.4809 | 0.5803 | 0.4089 | 0.7128 | 0.3756 |
| 3-D-Hydroxybutyrate | −0.4614 | −0.5678 | −0.2015 | −0.5044 | −0.4235 |
| Citrate | 0.0294 | −0.398 | −0.1174 | 0.104 | 0.2022 |
| Lactate | 0.5573 | 0.5532 | 0.4156 | 0.217 | −0.0842 |
| Pyruvate | 0.0595 | 0.3898 | −0.0901 | −0.48 | −0.6466 |
| Alanine | 0.459 | −0.2222 | −0.1705 | 0.419 | 0.412 |
| Glutamine | 0.8346 | −0.1312 | −0.3721 | −0.7205 | −0.7145 |
| N-acetyled-Glycoprotein | −0.2991 | −0.8376 | 0.5348 | 0.8351 | 0.8866 |
| Histidine | −0.0747 | −0.3349 | 0.0207 | 0.2849 | 0.3354 |
| Isoleucine | 0.7323 | −0.371 | −0.2441 | 0.3296 | 0.3508 |
| Lysine | 0.1145 | −0.3589 | −0.3768 | 0.22 | 0.2541 |
| Methionine | 0.2273 | −0.3984 | −0.4716 | 0.0594 | 0.0592 |
| Phenylalanine | 0.0186 | −0.6329 | −0.5822 | 0.1781 | 0.1437 |
| Taurine | −0.3313 | −0.4098 | −0.6389 | −0.1874 | −0.4622 |
| Threonine | 0.4466 | −0.2565 | −0.15 | 0.3202 | 0.338 |
| Tyrosine | 0.2934 | −0.2419 | −0.0929 | 0.3665 | 0.3639 |
| Valine | 0.9 | −0.2492 | −0.2122 | 0.3276 | 0.3066 |
| Glucose | −0.5492 | −0.1514 | −0.2495 | −0.4718 | −0.645 |
| | B—In Mature Mice vs. Normal Aging Mice | | | | |
| Lipid (0.85 L) | −0.3481 | −0.3772 | 0.3492 | 0.0298 | 0.1954 |
| Lipid (0.870) | −0.589 | −0.1169 | −0.1528 | −0.1362 | −0.1245 |
| Lipid (0.882) | −0.2826 | 0.3149 | −0.4563 | −0.1922 | −0.3849 |
| Lipid (1.3) | 0.0793 | −0.0343 | −0.1008 | 0.0622 | −0.2104 |
| Unsaturated Lipids (5.35) | −0.1346 | −0.2012 | −0.1858 | −0.0918 | −0.3599 |
| 3-D-Hydroxybutyrate | −0.4715 | −0.5034 | 0.2752 | −0.13 | −0.3183 |
| Citrate | 0.1902 | −0.3062 | 0.3744 | 0.2571 | 0.2258 |
| Lactate | 0.3873 | −0.0164 | 0.2546 | 0.2707 | 0.4091 |
| Pyruvate | 0.1781 | −0.0421 | 0.3371 | 0.4055 | 0.3486 |
| Alanine | 0.7018 | 0.599 | −0.277 | 0.0845 | 0.2498 |
| Glutamine | −0.206 | −0.0486 | −0.1919 | −0.0431 | −0.4195 |
| N-acetyled-Glycoprotein | 0.5279 | 0.4001 | 0.4719 | 0.5345 | 0.5668 |
| Histidine | 0.2439 | 0.6256 | 0.0303 | 0.1737 | −0.063 |
| Isoleucine | 0.6442 | 0.4829 | −0.255 | −0.0065 | 0.0813 |
| Lysine | 0.4945 | 0.5399 | −0.3204 | 0.0019 | 0.0939 |
| Methionine | 0.2657 | 0.4626 | −0.2395 | −0.0196 | 0.0307 |
| Phenylalanine | 0.3032 | 0.2814 | −0.2272 | −0.0663 | −0.1047 |
| Taurine | 0.0929 | 0.249 | −0.1421 | 0.1036 | −0.0271 |
| Threonine | 0.5097 | 0.5708 | −0.3066 | −0.0277 | −0.0131 |
| Tyrosine | 0.4757 | 0.5057 | −0.2691 | 0.0546 | −0.0246 |
| Valine | 0.7675 | 0.5593 | −0.3104 | 0.0553 | 0.1413 |
| Glucose | −0.6299 | −0.5065 | 0.0773 | −0.0024 | −0.078 |
| | C—In Senior Mice vs. Normal Aging Mice | | | | |
| Lipid (0.85 L) | −0.3058 | 0.2895 | −0.1632 | −0.0143 | −0.3684 |
| Lipid (0.870) | 0.2065 | 0.2869 | −0.0888 | 0.0925 | 0.1548 |
| Lipid (0.882) | 0.6625 | 0.245 | 0.098 | 0.2022 | 0.3579 |
| Lipid (1.3) | 0.4827 | 0.1621 | 0.1579 | 0.071 | −0.0968 |
| Unsaturated Lipids (5.35) | 0.5094 | 0.3094 | 0.0896 | 0.2477 | 0.2351 |
| 3-D-Hydroxybutyrate | −0.3542 | 0.0012 | −0.3872 | 0.138 | −0.1368 |
| Citrate | −0.6456 | −0.1823 | 0.2801 | 0.2395 | −0.3361 |
| Lactate | 0.304 | 0.3327 | 0.2786 | −0.0036 | 0.2288 |
| Pyruvate | −0.1064 | −0.4246 | −0.1083 | −0.3781 | −0.3693 |
| Alanine | 0.512 | 0.0913 | 0.1693 | −0.2526 | 0.3315 |
| Glutamine | −0.2677 | −0.68 | −0.1266 | −0.1752 | −0.5555 |
| N-acetyled-Glycoprotein | −0.154 | −0.1918 | 0.2935 | −0.2448 | −0.0994 |
| Histidine | −0.1372 | −0.3804 | −0.2225 | −0.2834 | −0.2441 |
| Isoleucine | 0.7706 | 0.3261 | 0.3659 | 0.1078 | 0.4926 |
| Lysine | 0.696 | −0.0481 | 0.2602 | 0.1645 | 0.4333 |
| Methionine | 0.0724 | −0.5774 | 0.0312 | −0.1541 | 0.2353 |
| Phenylalanine | 0.5845 | −0.2071 | −0.2666 | 0.1912 | 0.5293 |
| Taurine | 0.2083 | −0.4308 | −0.2838 | −0.023 | 0.3145 |
| Threonine | 0.2874 | −0.1882 | 0.0872 | −0.3998 | 0.3315 |
| Tyrosine | 0.5738 | −0.2234 | 0.3191 | 0.0546 | 0.4014 |
| Valine | 0.9021 | 0.1514 | 0.2872 | −0.205 | 0.3978 |
| Glucose | −0.7267 | −0.1577 | −0.2534 | 0.1972 | −0.1789 |

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims the invention may be practiced otherwise than as specifically described.

1. A dietary regimen useful for mimicking caloric restriction in an animal comprising administering to the animal:
a first diet comprising a caloric restriction mimicking amount of one or more dietary supplements capable of mimicking caloric restriction when the animal is a young animal;
a second diet comprising a caloric restriction mimicking amount of one or more dietary supplements capable of mimicking caloric restriction when the animal is an adult animal; and
a third diet comprising a caloric restriction mimicking amount of one or more dietary supplements capable of mimicking caloric restriction when the animal is a senior animal;
wherein the dietary supplements in the first diet, second diet, and third diet cannot all be the same dietary supplements.

2. The dietary regimen of claim 1 wherein the dietary supplements in each diet are different dietary supplements.

3. The dietary regimen of claim 1 wherein the dietary supplements in two diets are different dietary supplements.

4. The dietary regimen of claim 1 wherein the dietary supplements in the first diet are at least one of Ginkgo biloba and L-carnitine; the dietary supplements in the second diet are at least two of vitamin C, vitamin E, grape seed proanthocyanidin extract, and cysteine; and the dietary supplement in the third diet is L-carnitine.

5. The dietary regimen of claim 4 wherein the dietary supplement in the first diet is Ginkgo biloba.

6. The dietary regimen of claim 4 wherein the dietary supplement in the first diet is L-carnitine.

7. The dietary regimen of claim 4 wherein the dietary supplements in the second diet are vitamin C, vitamin E, grape seed proanthocyanidin extract, and cysteine.

8. The dietary regimen of claim 7 wherein the dietary supplement in the first diet is Ginkgo biloba.

9. The dietary regimen of claim 7 wherein the dietary supplement in the first diet is L-carnitine.

10. The dietary regimen of claim 7 wherein the dietary supplements in the first diet are Ginkgo biloba and L-carnitine.

11. The dietary regimen of claim 1 wherein the animal is a human.

12. The dietary regimen of claim 1 wherein the animal is a companion animal.

13. The dietary regimen of claim 12 wherein the animal is a dog or a cat.

14. The dietary regimen of claim 4 wherein ginkgo biloba is administered to the animal in amounts of from about 0.1 to about 10 mg/kg/day; L-carnitine is administered to the animal in amounts of from about 0.05 to about 20 mg/kg/day; vitamin C is administered to the animal in amounts of from about 0.5 to about 40 mg/kg/day; vitamin E is administered to the animal in amounts of from about 0.1 to about 20 International Units per day (IU)/kg/day; seed proanthocyanidin extract is administered to the animal in amounts of from about 10 to about 1000 mg/kg/day; and cysteine is administered to the animal in amounts of from about 6 to about 600 mg/kg/day, as appropriate depending on the choice of dietary supplements used in each diet.

15. The dietary regimen of claim 1 wherein the diets are formulated as a human food diet, pet food diet, nutraceutical diet, or a pharmaceutical diet.

16. The dietary regimen of claim 1 wherein the diets have at least one distinctive characteristic relative to at least one of the other diets.

17. The dietary regimen of claim 1 further comprising administering to the animal one or more of the first diet, second diet, and third diet to the animal in conjunction with one or more CR drugs in an amount effective for mimicking caloric restriction.

18. The dietary regimen of claim 17 wherein the CR drugs are administered with at least two of the diets.

19. The dietary regimen of claim 17 wherein the CR drugs are selected from the group consisting of resveratrol; metformin; endocannabinoid-1 receptor blockers; lipoic acids; 2-deoxy-D-glucose; mannoheptulose, leptin; peroxisome proliferator-activated receptor (PPAR) gamma modulators; and combinations thereof.

20. A dietary regimen useful for mimicking caloric restriction in an animal comprising administering to the animal:
a first diet comprising a caloric restriction mimicking amount of at least one of Ginkgo biloba and L-carnitine when the animal is a young animal;
a second diet comprising a caloric restriction mimicking amount of vitamin C, vitamin E, grape seed proanthocyanidin extract, and cysteine when the animal is an adult animal; and
a third diet comprising a caloric restriction mimicking amount of L-carnitine when the animal is a senior animal.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)
